(19) United States
(12) Patent Application Publication
(10) Pub. No.: US 2024/0262798 A1
(43) Pub. Date: Aug. 8, 2024

(54) A PROCESS AND PLANT FOR THE SYNTHESIS OF UREA AND MELAMINE

(71) Applicant: Casale SA, Lugano (CH)

(72) Inventors: Paolo Bertini, Lugano (CH); Matteo Fumagalli, San Fermo della Battaglia (IT)

(73) Assignee: Casale SA, Lugano (CH)

(21) Appl. No.: 18/577,854

(22) PCT Filed: Jun. 30, 2022
(86) PCT No.: PCT/EP2022/068150

§ 371 Date: Jan. 9, 2024

(30) Foreign Application Priority Data
Jul. 9, 2021 (EP) 21184757.9

(51) Int. Cl. C07D 251/60; C07C 273/04; C07C 273/12

(52) U.S. Cl. CPC C07D 251/60; C07C 273/04; C07C 273/12

(57) ABSTRACT

A process for the synthesis of urea and melamine, wherein urea synthesized in a urea plant is used to produce melamine in a melamine plant connected to the urea plant, according to a high-pressure non-catalytic melamine synthesis process, and offgas (16) liberated during the synthesis of melamine are returned to the urea plant after offgas processing (8) which includes a condensation and a urea formation step so that a urea-containing recycle stream (19) is returned to the synthesis section of the urea plant.

### FIELD OF THE INVENTION

The invention relates to the field of combined production of urea and melamine.

### PRIOR ART

Urea is produced industrially by reacting NH₃ and CO₂ at high pressure and high temperature. The reaction of ammonia and carbon dioxide produces ammonium carbamate which dehydrates to form urea and water. Due to the thermodynamic equilibrium of the reactions, the effluent of the reaction process is an aqueous solution of urea containing a significant amount of unconverted ammonia and carbon dioxide in the form of ammonium carbamate.

The state-of-the-art technology for the production of urea is the so-called stripping process wherein the reaction effluent is heated in a high-pressure stripper to decompose the ammonium carbamate into gaseous ammonia and carbon dioxide which are then removed from the solution. The vapours extracted from the stripper are condensed in a high-pressure condenser and the so obtained condensate stream is returned to the reactor.

The stripping process is normally performed in a vertically arranged shell-and-tube apparatus wherein the solution flows in the tubes of an externally heated tube bundle. To facilitate the removal of the gaseous ammonia and carbon dioxide, a stripping medium may be added. For example the CO2-stripping process uses gaseous CO2 introduced at the bottom of the stripper as a stripping medium. The so called self-stripping process uses no added stripping medium and the ammonia-stripping process uses gaseous ammonia.

The urea reactor, the high-pressure stripper and the high-pressure condenser operate substantially at the same pressure and form a so called urea synthesis loop or high-pressure loop. The loop pressure is typically well above 100 bar, for example around 150 bar or above. The high-pressure loop, particularly in the case of a CO2 stripping plant, may also include a high-pressure scrubber wherein a gas phase removed from the reactor is scrubbed with a recycle carbamate solution from the low-pressure stage.

The urea-containing aqueous solution effluent from the stripper is further processed in one or more recovery sections, for example a low-pressure recovery section or a medium-pressure recovery section followed by a low-pressure recovery section. A recovery section typically includes at least a carbamate decomposer and a condenser where vapours of ammonia and carbon dioxide are condensed to form a recycle solution. The so obtained recycle carbamate solution is pumped back to the high-pressure loop, e.g. into the high-pressure condenser.

The recovery section produces a purified urea solution, comprising urea, water and unavoidable impurities. This purified urea solution may be used as such or further processed to remove water, e.g. in an evaporation section, obtaining a highly concentrated solution or a so-called urea melt.

A more detailed overview of the urea production processes can be found in the relevant literature, for example in Meessen, “Urea”, Ullmann's Encyclopedia of Industrial Chemistry, 2012.

Urea has several industrial uses including the production of fertilizers, the production of melamine, and other more recent applications such as the production of additives for selective catalytic reduction of NOx in the exhaust gas treatment.

Melamine can be produced from urea with a low-pressure catalytic process or, preferably, with a high-pressure non-catalytic process. These processes for the synthesis of melamine are familiar to a skilled person. The high-pressure non-catalytic process, which is nowadays preferred, operates at a pressure of 70 bar or above, preferably 100 bar or above.

The integration of a urea plant with a melamine plant is attractive because on one side, urea is the starting material for the synthesis of melamine, on the other side, the synthesis of melamine produces a gaseous stream composed predominantly of ammonia and carbon dioxide (melamine offgas) which can be recycled to the urea plant to produce more urea.

The melamine offgas may be returned to the urea plant either directly in gaseous form or after condensation. In the melamine high-pressure non-catalytic process, the melamine offgas are released at a high pressure and may be substantially anhydrous. In that case, the condensation of the gas and the recycle in a liquid form are considered particularly attractive. The condensation of the melamine offgas is performed with the help of a carbamate solution or ammoniacal solution to reduce the risk of crystallization, and recycle the so obtained condensate to the urea plant.

A plant and a process for production of urea and melamine with recycle of the melamine offgas is described for example in WO 2015 165 741.

Recycling the melamine offgas via condensation is considered a preferred solution because the condensation heat may be used to produce steam, typically at a pressure around 3-4 bar and the liquid condensate can be easily raised to the urea synthesis pressure with a pump or an ejector. Therefore, the condensation of the offgas is normally performed to condense as much as possible of the gas (total condensation).

However, the above mentioned integration of the urea and melamine production has still some drawbacks.

First, the offgas total condensation produces steam at a relatively low pressure: as above mentioned, steam may be at 1 to 5 bar and more often at 3 to 4 bar. Producing steam at a higher pressure would be desirable.

A second and more important problem is that the urea plant may not be able to fully exploit the additional amount of reagents coming from the offgas. For example a melamine plant may be added to, and integrated with, an existing urea plant. In that case, the urea plant may not be able to process the additional amount of reagents, particularly in the high-pressure synthesis section. Revamping the high-pressure to a higher capacity is expensive and may render the entire operation—of modernizing a urea plant into a urea-melamine integrated plant—less attractive.

EP 1 752 447 discloses a process for the integrated production of urea and melamine. WO 2019/145169 discloses a plant for the synthesis of melamine with off-gas recover in a tied-in urea plant.

### SUMMARY OF THE INVENTION

The invention aims to overcome the above mentioned drawbacks of the prior art concerning the integration between the production of urea and the production of melamine.

This aim is reached with a process and plant according to the claims.

In the inventive process, the melamine offgas are returned to the synthesis section of the urea plant in the form of a urea-containing stream obtained after offgas processing which includes a condensation step and a urea formation step.

Accordingly, a plant for the combined production of urea and melamine includes a melamine offgas condensation section having at least one reaction environment configured to operate under urea forming conditions, and a line arranged to return the so obtained urea-containing recycle stream to the urea synthesis section.

### DESCRIPTION OF THE INVENTION

The invention is applicable to a plant for the combined production of urea and melamine. Such plant is understood as a plant wherein urea is produced; at least part of said urea is used to produce melamine, and the melamine offgas are returned to the urea plant for use in the urea synthesis. Such plant is also termed an integrated plant for urea and melamine production.

The invention is based on the intuition that the processing of the melamine offgas can be regarded as a further opportunity to produce urea. In contrast with the prior art wherein the goal of the melamine offgas processing is to produce a carbamate containing recycle stream, the present invention aims to produce additional urea from carbon dioxide and ammonia contained in the offgas stream, so that the stream returned to the urea plant is a urea-containing stream. A further intuition behind the invention is that heat released during the condensation of ammonia and carbon dioxide contained in the melamine offgas can be used to reach a urea-forming temperature during the offgas processing.

Accordingly the offgas extracted from the melamine plant are sent to a processing section which includes a urea synthesis reaction environment operated under urea-forming conditions and having a suitable volume to produce urea.

The invention provides an additional urea-forming environment wherein urea is synthesised from the ammonia and carbon dioxide contained in the offgas, prior to the recycle to the urea synthesis section. Hence an additional urea synthesis capacity is provided. A remarkable advantage is de-bottlenecking the high-pressure urea synthesis section, such as high-pressure loop, of the urea plant. This is of particular interest in the context of a revamping process including the integration of an existing urea plant with a melamine plant. The existing urea synthesis section may not have a significant spare capacity, thus with a conventional approach it may not benefit, or benefit only partially, from the recycle of melamine offgas. Thanks to the invention, the ammonia and carbon dioxide contained in the melamine offgas are exploited to increase the urea production capacity.

The urea-containing recycle solution may be fed to different items of the urea synthesis section of the urea plant. In a preferred embodiment, said recycle solution is sent to a high-pressure carbamate condenser of the urea plant. In that case, an additional advantage is obtained because said carbamate condenser, thanks to the recycle of the urea containing solution from off-gas condensation, can condense the vapours from the high-pressure stripper at a higher temperature, thus producing steam at a higher pressure, or the condenser may deliver a greater duty without the need of internal modification.

Said high-pressure carbamate condenser may be part of a high-pressure synthesis loop whose main items are a reactor, a stripper and optionally a scrubber.

As mentioned above, in the combined production of urea and melamine at least part of the urea is used to produce melamine. The urea used to produce melamine is normally a urea melt obtained by removing water from the purified aqueous solution produced in the recovery section. Water can be removed in a suitable evaporation section. In an embodiment, all the urea produced in the urea plant is used to produce melamine in the tied-in melamine plant. In some embodiments, part of the urea is used for a different purpose, e.g. sent to a finishing section to produce solid granules or prills of urea, and/or for other uses such as the production of a water-urea solution for use in SCR systems for removal of NOx.

In the present invention, melamine is produced with the high-pressure non catalytic process, which is known to a skilled person. In this process, melamine is synthesised at a pressure which is typically of at least 70 bar, more preferably above 100 bar. Accordingly, the melamine offgas may be extracted at a high pressure, which is about the same as the melamine synthesis pressure, and fed to the melamine offgas condensation section at said high pressure. The pressure of the melamine offgas at the inlet of the offgas condensation section may be substantially same as the melamine synthesis pressure, which means the same pressure minus the pressure drop inherent to transporting the offgas from the melamine plant to the condensation section, caused by piping and ancillary equipment such as flow control valves.

Particularly preferably, the melamine offgas are free of water or substantially free of water. Preferably the melamine offgas contain no more than 1% of water in weight. An advantage of anhydrous offgas is that no water is introduced in the urea synthesis section wherein water shifts the chemical equilibrium against the formation of urea.

In a preferred embodiment the condensation of melamine offgas is performed at a pressure of at least 90 bar, preferably at least 120 bar. Even more preferably said condensation of the melamine offgas is performed at a pressure in the range 120 to 150 bar, for example 130 to 150 bar or 140 to 150 bar.

The pressures in this description are given in bar gauge, i.e. they denote the pressure relative to atmospheric pressure conventionally set at 101325 Pa.

The amount of urea formed in the melamine offgas condensation section is a non-negligible amount which may vary according to the case; preferably it is at least 5% by weight, more preferably at least 10% by weight, of the total amount of urea which is synthesized in the combined plant. More preferably the urea produced in the offgas condensation section is 10% to 30% by weight of the total, for example 10% to 20%.

The urea-containing recycle stream, which is obtained from the offgas processing, contains preferably at least 12% by weight of urea, and more preferably at least 20% by weight. Said content of urea is normally up to 40% or up to 30% by weight. In a preferred embodiment said recycle stream may contain 12% to 40% or 20% to 40% by weight of urea.

The condensation of the offgas is preferably a partial condensation. A partial condensation means that condensation is deliberately performed only partially. Hence the recycle stream returned to the synthesis section of the urea plant contains some uncondensed offgas. This is also in contrast with the teaching of the prior art which tends to provide a full condensation of the melamine offgas, i.e. apart from incondensables and inevitable small amounts of uncondensed vapours.

A related advantage is that the offgas condensation section is able to produce steam at a higher pressure compared to the prior art. A further advantage of the above described partial condensation is that a further condensation of the offgas can be performed in the synthesis section of the urea plant, particularly in the urea synthesis reactor. This further condensation releases enthalpy to heat the reactor and helps maintain a temperature suitable for the formation of urea, typically above 180° C. Hence according to another feature of the invention the degree of partial condensation during offgas processing can be adjusted and controlled according to the heat required in the urea synthesis reactor.

The condensate stream obtained after the offgas condensation therefore may be a two-phase flow.

The urea formation step during offgas processing is performed preferably under the following conditions: a nitrogen-to-carbon N/C ratio in the range 2.8 to 5.0, preferably 2.9 to 4.0, and/or a hydrogen-to-carbon H/C ratio in the range 0.2 to 2.0, preferably 0.4 to 1.0.

More preferably the offgas condensation is performed in the presence of at least one stream of an aqueous solution withdrawn from the urea plant or from the melamine plant, and optionally in the presence of an added stream of ammonia. Said aqueous solution is preferably a stream of carbamate solution which is recycled from a recovery section of the urea plant. In a preferred embodiment, said aqueous solution and/or ammonia may be added to the offgas stream before entering a condensation section or brought into contact with the melamine offgas during their condensation.

Said carbamate solution may be obtained from the urea recovery process, namely from a medium-pressure or a low-pressure recovery section of the urea plant. Adding an aqueous solution, such as carbamate solution, may facilitate the condensation process and furthermore controlling the amount of said solution, and consequently the amount of water added to the offgas, may be used to control the hydrogen to carbon (H/C) ratio during formation of urea. Conversely, the addition of ammonia may control the N/C ratio.

Therefore the addition of a controlled amount of a recycle aqueous solution and ammonia may represent a means to control the N/C ratio and H/C ratio within the above mentioned target ranges. Hence a further preferred feature is a process including the step of controlling said N/C ratio by means of controlling the amount of added solution and/or controlling the H/C ratio by means of controlling and the amount of added ammonia.

The condensation process is normally performed by removing heat from the offgas, which is transferred to a cooling medium, typically water to produce steam. In certain embodiments however, the added aqueous solution may be sufficient to reach the desired degree of partial condensation of the offgas. In such a case the partial condensation may be performed without transferring heat to another medium by indirect heat exchange.

The offgas processing includes a condensation step and a urea formation step. Said steps may be performed in the same environment or preferably in different environments of an offgas processing section, such as a condensation environment and a reaction environment in fluid communication. Said condensation environment and reaction environment may be hosted in a single pressure vessel or in separate vessels.

In a preferred embodiment the offgas processing is performed in a melamine offgas processing section and includes a condensation step which is performed in a condensation environment, obtaining a condensate flow which is then transferred to a urea reaction environment where urea is formed thus obtaining said urea-containing recycle stream.

Said offgas processing section is not part of the urea synthesis section of the urea plant (e.g. high-pressure synthesis loop). Normally the offgas processing section operates at a lower pressure than said urea synthesis section.

The processing of the melamine offgas is preferably isobaric. Accordingly, the partial condensation and the urea formation are performed at the same pressure or substantially the same pressure. The term of substantially the same pressure refers to the same pressure apart from differences due to the transport of fluids from the condensation environment to the reaction environment.

In an embodiment, the urea synthesis reaction environment is hosted in a separate vessel or reactor, which is preferably vertical. This reactor vessel can be fitted with suitable internals to promote the formation of urea, for example perforated plates.

In another embodiment the offgas processing section includes a pool condenser. A pool condenser is typically a horizontal shell-and-tube heat exchanger where, in the shell side, the offgas are condensed and a proper liquid level is maintained under urea forming conditions, so that the condensate effluent contains urea. Accordingly, a pool condenser may perform the partial condensation of the offgas and provide the formation of urea, producing the urea-containing stream to be returned to the urea plant.

The urea-containing stream may be transferred from the melamine offgas condensation section to the urea synthesis section via a buffer vessel. In the buffer vessel, a suitable amount of condensate stream is stored under pressure, to compensate for fluctuations of the process. More preferably vapours are removed from the buffer vessel and said vapours are subject to a washing step e.g. in a washing column. Said washing column may be integrated in the buffer vessel, i.e. a single pressure vessel may provide the required storage volume and washing column. Alternatively the buffer may be integrated in a reactor of the offgas processing section.

Very preferably, the urea plant operates according to a stripping process, such as CO2-stripping process, self-stripping process or ammonia-stripping process. The urea stripping process is well known from the literature and needs not be described in detail. A plant designed to implement the urea stripping process typically comprises a high-pressure synthesis loop including a reactor, a stripper, a high-pressure carbamate condenser and optionally a scrubber. In a preferred embodiment, the urea-containing recycle solution is fed to said high-pressure carbamate condenser.

In common embodiments the pressure in the urea synthesis loop is greater than the pressure in the melamine offgas condensation section, and therefore the urea-containing solution must be raised to a suitable pressure for its recycle. This can be made with a suitable pump or an ejector. An ejector may be used if a motive stream at a sufficiently high pressure is available. Said motive stream may be a stream of fresh ammonia feed.

In the reaction environment, which may be a separate reactor as above disclosed, the conversion may be quite high and even above 50% under favourable conditions. For example assuming the offgas are at a pressure of at least 120 bar, N/C is about 3.0 and H/C is 0.4 to 0.6, a conversion rate greater than 50% is achievable.

The invention includes also a plant for the combined production of urea and melamine according to the claims.

### DESCRIPTION OF THE FIGURES

The invention is further elucidated with the help of the figures wherein:

FIG. 1 is a scheme of a urea-melamine process and plant according to an embodiment of the invention.

FIG. 2 is a scheme of an offgas condensation section according to a preferred embodiment.

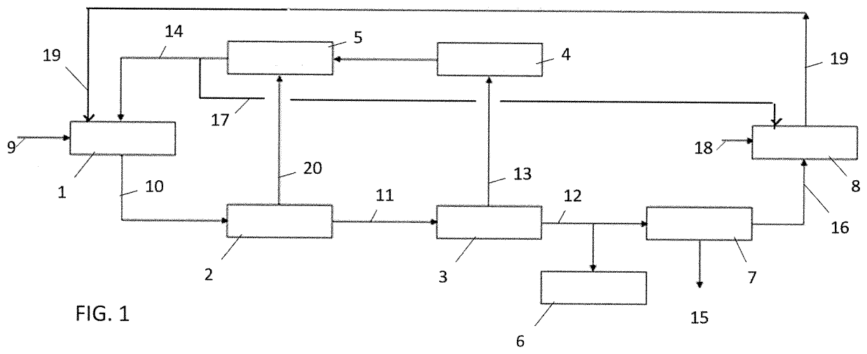

FIG. 1 illustrates the following blocks, whose understanding is easy for a skilled person. Each of the blocks in FIG. 1 may be regarded as a process step or a corresponding section of the plant performing the process step.

The block 1 denotes a urea synthesis step at a urea synthesis pressure. The block 1 accordingly denotes also a urea synthesis section, such as a synthesis loop including a reactor, a stripper and a condenser forming a high-pressure loop. This step receives fresh reagents generally denoted by the input line 9 and delivers a solution 10 comprising urea, water and unconverted ammonia and CO2.

The block 2 denotes a step of carbamate decomposition which is performed for example in one or more recovery sections. Here the solution 10 is purified to give an aqueous solution 11 made of urea, water and unavoidable impurities. A vapour stream 20 comprising ammonia, CO2 and water vapour is separated.

The block 3 denotes an evaporation step wherein water is removed from the urea solution 11 to provide a urea melt 12. This can be made in a suitable evaporation section, by heating the solution and/or reducing pressure under vacuum (flash). The water vapour 13 removed from the solution, which is contaminated with some ammonia and CO2, is sent to a condensate treatment step 4.

The block 5 denotes a recycling section which receives the vapours 20 from the recovery section 2 and the condensate stream from the condensate treatment section 4. Here, the vapours are condensed to produce a recycle carbamate solution and sent back to the urea synthesis section 1 via line 14. This step of vapours condensation is performed typically at a medium pressure.

A first portion of the urea melt 12 is sent to a finishing step 6, for example in a granulator or prilling tower, to produce solid urea.

A second portion of the urea melt 12 is sent to a high-pressure melamine synthesis step 7 producing melamine 15. Offgas 16, predominantly made of ammonia and CO2, are also produced.

Said offgas are recycled to the urea process via an offgas processing section 8 which receives also a portion of recycle carbamate solution via line 17 and a feed of fresh ammonia via line 18.

Said offgas processing section 8 includes a urea synthesis environment under urea forming conditions so that its effluent 19 contains ammonia, carbon dioxide (possibly in the form of ammonium carbamate), water and urea. Said effluent is sent back to the urea synthesis section 1. More specifically, in the processing section 8 some urea is obtained from the ammonia and CO2 contained in the offgas stream 16, thus providing an additional capacity for the synthesis of urea.

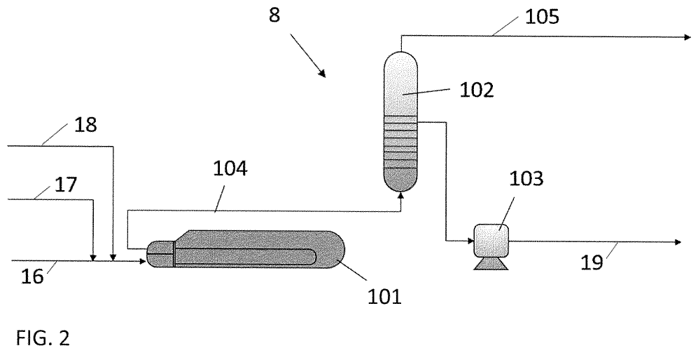

FIG. 2 illustrates an embodiment of the offgas processing section 8 including a shell-and-tube condenser 101 and a reactor 102 in separate vessels. Here, the condenser 101 provides a condensation environment while the reactor 102 provides a urea synthesis reaction environment.

The condenser 101 receives the offgas 16 mixed with the carbamate solution 17 and the ammonia stream 18. This mixture is partially condensed passing through the tube side of the condenser 101, and heat of condensation is transferred to water/steam in the shell side.

The so obtained condensed stream 104, which may be a biphasic stream, is sent to the reactor 102. In the reactor 102, the mixture is maintained under urea forming conditions so that urea is formed and the urea-carbamate stream 19 is obtained. A pump 103 raises the urea-carbamate stream to a suitable pressure for recycle to the high-pressure section 1.

The provision of said pump 103 is not mandatory, but depends on the pressure difference between melamine and urea synthesis section. Generally, melamine synthesis operates at lower pressure than urea synthesis, hence the need for the pump.

FIG. 2 also illustrates vapours 105 removed from the reactor 102, which may be sent for example to the recycle section 5 for condensation at a medium pressure. Said vapours 105 may be washed in a suitable washing column prior to recycle. The washing column may be part of the reactor 102 or a separate vessel.

In another embodiment the condenser 101 and the reactor 102 may be combined in a single apparatus.

A buffer vessel is preferably provided on the line 19. A suitable buffer capacity may also be integrated within the reactor 102.

1) A process for the synthesis of urea and melamine, wherein urea synthesized in a urea plant is used to produce melamine in a melamine plant connected to said urea plant, according to a high-pressure non-catalytic melamine synthesis process, and offgas liberated during the synthesis of melamine are returned to the urea plant after offgas processing which includes a condensation step, wherein said offgas processing further includes a urea formation step and produces a urea-containing recycle stream which is returned to a urea synthesis section of said urea plant.

2) The process according to claim 1, wherein condensation of melamine offgas is performed at a pressure of at least 90 bar.

3) The process according to claim 1, wherein the urea formed during offgas processing is at least 5% by weight of the total amount of synthesized urea, and/or wherein said urea-containing recycle stream contains at least 12% by weight of urea.

4) The process according to claim 1, wherein the condensation of the offgas, which is performed during offgas processing, is a partial condensation.

5) The process according to claim 1, wherein the urea formation step during offgas processing is performed under a N/C ratio in the range of 2.8 to 5.0, and a H/C ratio in the range of 0.2 to 2.0.

6) The process according to claim 1, wherein the offgas condensation is performed in the presence of at least one stream of an aqueous solution withdrawn from the urea plant or from the melamine plant, and optionally in the presence of an added stream of ammonia.

7) The process according to claim 5, further including the step of controlling said H/C ratio by means of controlling the amount of said aqueous solution and/or controlling the N/C ratio by means of controlling the amount of added ammonia.

8) The process according to claim 1, wherein the offgas processing is performed a melamine offgas processing section and includes a step of partial condensation of the melamine offgas, which is performed in a condensation environment of said melamine offgas processing section and in the presence of an aqueous solution, such as a carbamate-containing recycle stream, obtaining a two-phase condensate flow which is then transferred to a urea reaction environment of said melamine offgas processing section, where urea is formed thus obtaining said urea-containing recycle stream.

9) The process according to claim 1, wherein the offgas processing is an isobaric process wherein partial condensation of the offgas and formation of urea take place at the same pressure or substantially the same pressure.

10) The process according to claim 1, wherein urea is produced in the urea plant according to a stripping process, such as CO2-stripping process, self-stripping process or ammonia-stripping process.

11) The process according to claim 1, wherein said urea-containing stream is transferred to the urea plant via a buffer vessel, and wherein vapours are removed from the buffer vessel and said vapours are subject to a washing step and then recycled to the urea plant separately from the urea-containing solution.

12) The process according to claim 1, wherein:
a) in the urea plant, urea is synthesized from ammonia and carbon dioxide in a urea synthesis section operating at a urea synthesis pressure to form a urea-containing effluent,
b) said urea-containing effluent is processed at one or more pressure levels, which are lower than said urea synthesis pressure, to recover unconverted ammonia and carbon dioxide in the form of a recycle carbamate solution and to obtain a purified solution of urea;
c) at least part of the urea contained in said purified solution is used to produce melamine in the melamine plant;
d) melamine offgas withdrawn from the melamine plant are processed in an offgas processing section including a condensation section wherein the offgas are partially condensed, obtained a two-phase condensate flow, and a reaction section wherein the so obtained condensate flow is maintained under urea forming conditions so that urea is formed and the urea-containing recycle stream is produced,
e) the urea-containing recycle stream obtained from the offgas processing is recycled to said urea synthesis section.

13) A plant for the combined production of urea and melamine wherein:
the combined plant includes a urea plant and a tied-in melamine plant,
the urea plant includes a high-pressure urea synthesis section arranged to synthesise urea from ammonia and carbon dioxide and to produce a urea-containing effluent is produced;
the urea plant further includes at least one recovery section arranged to process said effluent to recover unconverted ammonia and carbon dioxide in the form of a recycle carbamate solution and to obtain a purified solution of urea;
a urea feed line is arranged to transfer at least part of the urea produced in the urea plant to the tied-in melamine plant, as a source material to produce melamine, the melamine plant being configured to produce melamine according to a high-pressure non-catalytic melamine synthesis process;
the combined plant further comprises a melamine offgas processing section and an offgas line arranged to collect the melamine offgas containing ammonia and carbon dioxide, which are liberated during the synthesis of melamine, and to feed said offgas to said melamine offgas processing section;
said melamine offgas processing section is configured to provide a recycle stream;
the combined plant further comprises a line arranged to return said recycle stream to the urea synthesis section;
wherein said melamine offgas processing section includes at least one reaction environment which is configured to operate under urea forming conditions so that urea is formed during offgas processing and said recycle stream, which is returned to the urea synthesis section, is a urea-containing stream.

14) The plant according to claim 13, wherein said melamine offgas processing section includes an offgas condensation section.

15) The plant according to claim 14, the plant further including a line arranged to feed a carbamate recycle solution from the urea recovery section to said offgas condensation section, and/or including a line arranged to feed gaseous ammonia to said offgas condensation section.

16) The plant according to claim 13 wherein said condensation section is configured so that the offgas are partially condensed and the offgas processing section includes a reaction section where urea is formed, and wherein said condensation section and said reaction section are hosted in the same pressure vessel or in separate pressure vessels.

17) The process according to claim 2, wherein condensation of melamine offgas is performed at a pressure of at least 120 bar.

18) The process according to claim 3, wherein the urea formed during offgas processing is at least 10% by weight of the total amount of synthesized urea, and/or wherein said urea-containing recycle stream contains at least 20% by weight of urea.

19) The process according to claim 5, wherein the urea formation step during offgas processing is performed under a N/C ratio in the range of 2.9 to 4.0, and a H/C ratio in the range of 0.4 to 1.0.

20) The process according to claim 3, wherein the urea formed during offgas processing is at least 10% by weight of the total amount of synthesized urea, and/or wherein said urea-containing recycle stream contains at least 20% to 40% by weight of urea.
